(19) United States
(12) Patent Application Publication
(10) Pub. No.: US 2004/0091481 A1
(43) Pub. Date: May 13, 2004

(54) Method for modulating the binding activity of a novel icam-3 binding receptor on sinusoidal endothelial cells in liver and lymph nodes

(76) Inventors: Carl Gustav Figdor, Den Bosch (NL); Teunis Bernard Herman Geitjenbeek, Amstelveen (NL); Yvette Van Kooyk, Amsterdam (NL); Ruurd Torensma, Nijmegen (NL)

Correspondence Address: Stephen A Saxe, Alexion Pharmaceuticals, 352 Knotter Drive, Cheshire, CT, 06410, US

(21) Appl. No.: 10/451,459

(22) PCT Filed: Dec. 21, 2001
(86) PCT No.: PCT/EP01/15392

(30) Foreign Application Priority Data
Dec. 21, 2000 (EP) 00128143.5
Mar. 13, 2001 (EP) 01200944.5

(51) Int. Cl. A61K 39/395; C07K 16/18

(52) U.S. Cl. 424/145.1; 530/388.25; 530/370

(57) ABSTRACT

The invention relates to the use of a compound that binds to a C-type lectin on the surface of a sinusoid endothelial layer, in the preparation of a composition for modulating, in particular reducing, the immune response in animal, in particular a human or another mammal. The sinusoid endothelial layer may be either constituted by liver sinusoid endothelial cells (LSEC) or by the lymph node sinusoidal zone.

The present invention relates to the use of a compound binding to a C-type lectin located on the surface of sinusoid endothelial cells in liver and lymph nodes for modulating the immune response in animals.

The molecule DC-SIGN has recently been identified as a DC-specific adhesion receptor that mediates the interaction between DCs and resting T cells through high affinity binding to ICAM-3, thereby facilitating the initiation of primary immune responses. DC-SIGN was shown to be identical to the previously reported type II membrane-associated C-type lectin (Geijtenbeek, T. B. et al., 2000, Cell 100:575-585) that binds HIV-1 envelope glycoprotein gp120 in a CD4-independent manner. The affinity of DC-SIGN exceeds that of CD4 for HIV-1 gp120 (Curtis, B. M. et al., 1992, Proc Natl Acad Sci USA 89:8356-8360), and upon capture of HIV-1, DC-SIGN does not appear to promote viral entry into the DC itself, but rather enhances infection of T cells in trans (Geijtenbeek, T. B. et al., 2000, Cell 100:587-597). DC-SIGN-associated HIV-1 remains infectious over a prolonged period of time, perhaps contributing to the infectious potential of the virus during its transport by DCs from the periphery to lymphoid organs.

A previous search by Yokoyama-Kobayashi et al. (1999, Gene 228:161-167) for cDNA clones encoding type II membrane proteins resulted in the identification of a partial clone that was homologous, but not identical to the cDNA encoding the molecule now known as DC-SIGN. The putative protein product contained a deletion of 28 amino acids in the cytoplasmic domain and was lacking the entire C-type lectin domain relative to the cDNA encoding DC-SIGN.

More recently, Soilleux et al. (2000, J Immunol 165:2937-2942) described the full length cDNA-sequence of the related gene, which they called DC-SIGNR. The genomic organization of DC-SIGN and DC-SIGNR was compared, indicating a high degree of similarity. Concomitant expression of the two genes in placenta, endometrium, and stimulated KG1 cells (a cell line that phenotypically resembles myeloid DCs) was observed, although the expression of DC-SIGNR was very low in both endometrium and stimulated KG1 cells.

In the research that led to the present invention it was now found that the DC-SIGNR gene is expressed at considerably high levels in only two tissues, liver and lymph node, but not in monocyte derived dendritic cells. The receptor was renamed “L-SIGN” because it is a liver/lymph node-specific ICAM-3 grabbing nonintegrin.

The homologous human C-type lectins DC-SIGN and L-SIGN appear to be the products of a recent gene duplication. The corresponding proteins share the same domain organization and overlapping, if not completely identical, ligand specificity. The most diverse region of these molecules occurs in their cytoplasmic tails.

Another obvious difference between the genes for DC-SIGN and L-SIGN is the repeat polymorphism in exon 4 of L-SIGN, which is conserved in DC-SIGN (Table 1). The neck domain of L-SIGN may contain from three to nine repeats while DC-SIGN always consisted of seven repeats among the Caucasians tested. No difference was observed between L-SIGN molecules containing six or L-SIGN molecules containing seven repeats in ligand binding, nor HIV-1 capture and enhancement experiments.

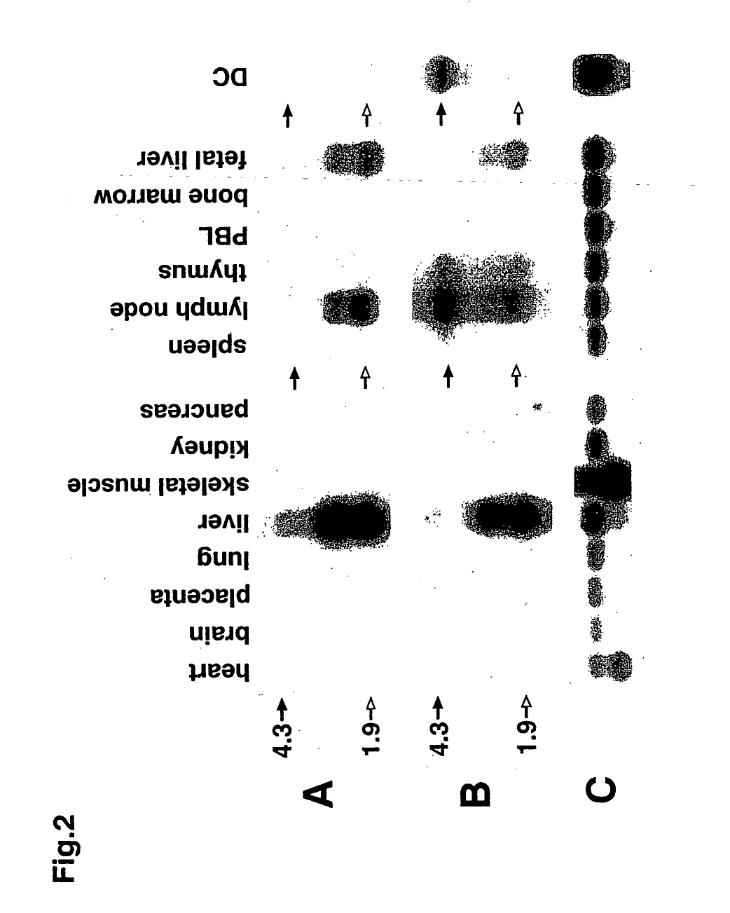

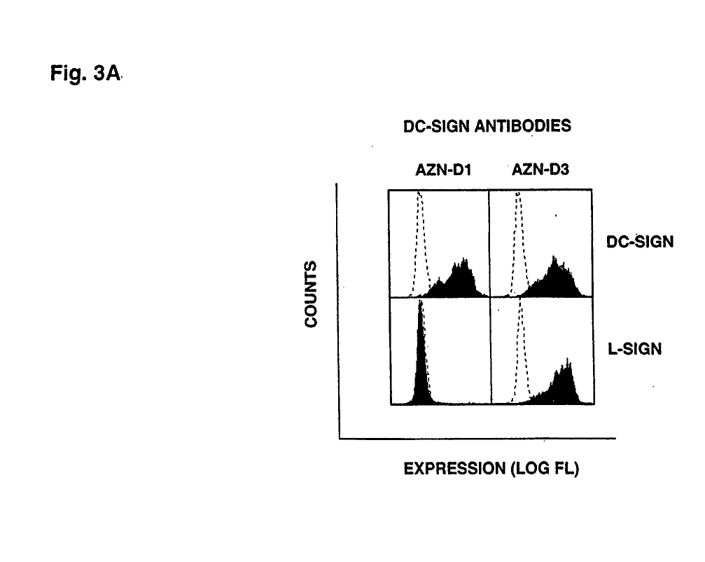

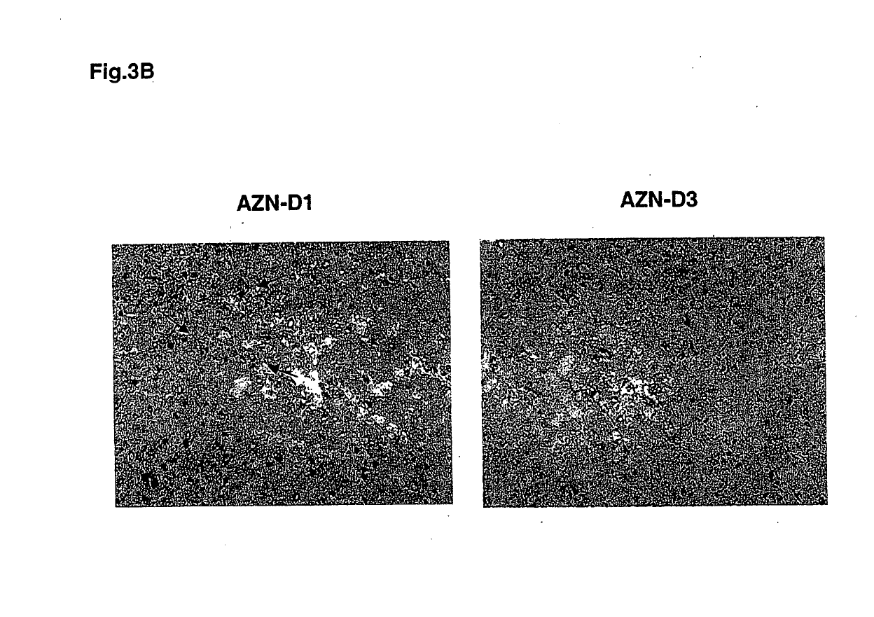

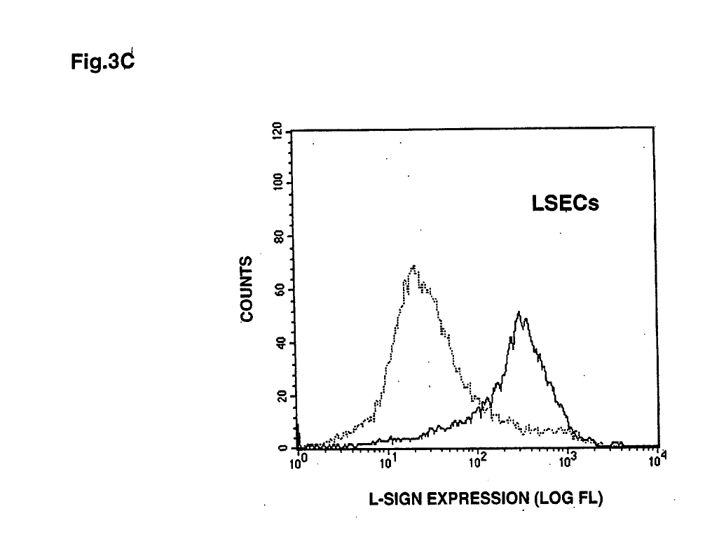

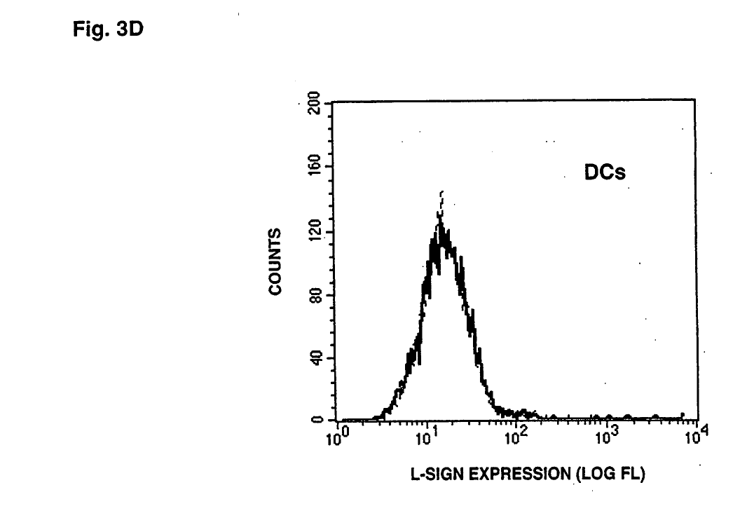

Although the SIGN genes have maintained sequence and functional similarity over their evolutionary history, it was now surprisingly found according to the invention that regulatory elements determining their tissue distribution have evolved along unique paths. Northern analysis of mRNA expression clearly-indicated expression of DC-SIGN in monocyte-derived DCs and in tissues where DCs reside, whereas expression of L-SIGN in DCs was undetectable (FIG. 2). Further, L-SIGN was not detected on monocyte-derived DCs using antibodies specific to L-SIGN (FIG. 3C). Thus, it was found that unique cell types in the lymph node express one, but not both SIGN molecules: L-SIGN is expressed by endothelial cells, as it is in liver, while DC-SIGN is expressed by DCs in T cell area of lymph node. This difference in expression pattern could not be expected based on the sequence homology.

Liver sinusoids are specialized capillary vessels characterized by the presence of resident macrophages adhering to the endothelial lining. The LSEC-leukocyte interactions, which require expression of adhesion molecules on the cell surfaces, constitute a central mechanism of peripheral immune surveillance in the liver. The mannose receptor as well as other costimulatory receptors such as MHC class II, CD80, and CD86 are known to be expressed on LSECs and to mediate the clearance of many potentially antigenic proteins from the circulation in a manner similar to DCs in lymphoid organs.

The inventors established that L-SIGN fits in this category of receptors on LSECs, as its tissue location and ligand binding properties strongly implicate a physiologic role for this receptor in antigen clearance, as well as in LSEC-leukocyte adhesion. The high expression of ICAM-3 on apoptotic cells are the means by which these cells are trapped by L-SIGN-expressing cells in the liver and subsequently cleared.

Like DC-SIGN, L-SIGN is a membrane-associated lectin that enhances HIV-1 infection. The expression of L-SIGN in liver sinusoids indicates that LSECs, which are in continual contact with passing leukocytes, capture HIV-1 from the blood and promote trans-infection of T cells.

In addition, LSECs themselves may be susceptible to HIV-1 infection. Thus, it is possible that L-SIGN promotes infection of these cells thereby establishing a reservoir for production of new virus to pass on to T lymphocytes trafficking through the liver sinusoid.

Based on the above observations, the present invention relates to the use of a compound that binds to a C-type lectin on the surface of cells of a sinusoid endothelial layer, in the preparation of a composition for modulating, in particular reducing, the immune response in a animal, in particular a human or another mammal. The C-type lectin on the surface of cells of a sinusoid endothelial layer is in particular L-SIGN.

The cells of the sinusoid endothelial layer may either be constituted by liver sinusoid endothelial cells (LSEC) or cells of the lymph node sinusoidal zone.

The composition of the invention may be used for modulating, in particular reducing, one or more interactions between a cell of a sinusoid endotlielial layer, in particular a LSEC, and a cell expressing ICAM-2 and/or ICAM-3, in particular a T cell. More in particular, the composition is used for modulating, in particular reducing, the adhesion between a cell of a sinusoid endothelial layer, in particular a LSEC, and a cell expressing ICAM-2 and/or ICAM-3, in particular a T cell, in particular between a C-type lectin on the surface of a LSEC and an ICAM receptor on the surface of a T cell, in particular an ICAM-2 or ICAM-3 receptor on the surface of a T cell.

The composition prepared according to the invention is applied for preventing or inhibiting immune responses to specific antigens, for inducing tolerance, for immunotherapy, for immunosuppression, for the treatment of autoimmune diseases, and/or for the treatment of allergy.

According to a further aspect thereof, the invention relates to the use of a compound that binds or can bind to a C-type lectin on the surface of a cell of the sinusoid endothelial layer, in particular a LSEC, in the preparation of a composition for inhibiting the HIV infection of cells of a sinusoid endothelial layer, in particular LSECs, in particular for inhibiting the adhesion of HIV surface protein (i.e gp120) to the surface of a cell of a sinusoid endothelial layer, in particular a LSEC and thereby the entry of HIV into said cell.

The invention furthermore relates to the use of a compound that binds or can bind to a C-type lectin on the surface of a cell of a sinusoid endothelial layer, in particular a LSEC, in the preparation of a composition for inhibiting the transfer of HIV from cells of a sinusoid endothelial layer (that may or may not be infected themselves), in particular a LSEC, to non-infected T cells.

Alternatively, the invention provides the use of a combination of: 1) a compound that binds to a C-type lectin on the surface of a cell of a sinusoid endothelial layer, in a particular a LSEC; and attached thereto: 2) an antigen or a fragment or part thereof; in the preparation of a composition for modulating, in particular generating, increasing and/or promoting, an immune response in an animal, in particular a human or other mammal, against said antigen. Preferably, the antigen is covalently bonded to or fused with the compound that can bind to the C-type lectin. The antigen is for example chosen from cancer antigens which can be used to generate an immune response against tumor cells that contain or express said antigen, or antigens as used in vaccines against infectious diseases.

The compound that can bind to a C-type lectin on the surface of a cell of a sinusoid endothelial layer, in particular a LSEC, is preferably chosen from the group consisting of mannose carbohydrates, such as mannan and D-mannose; fucose carbohydrates, such as L-fucose; plant lectins such as concanavalin A; antibiotics, such as pradimicin A; sugars such as N-acetyl-D-glucosamine and galactose; proteins such as gp120 and analogs or fragments thereof; and antibodies directed against a C-type lectin as expressed on the surface of a cell of a sinusoid endothelial layer, in particular a LSEC, or a part, fragment or epitope thereof.

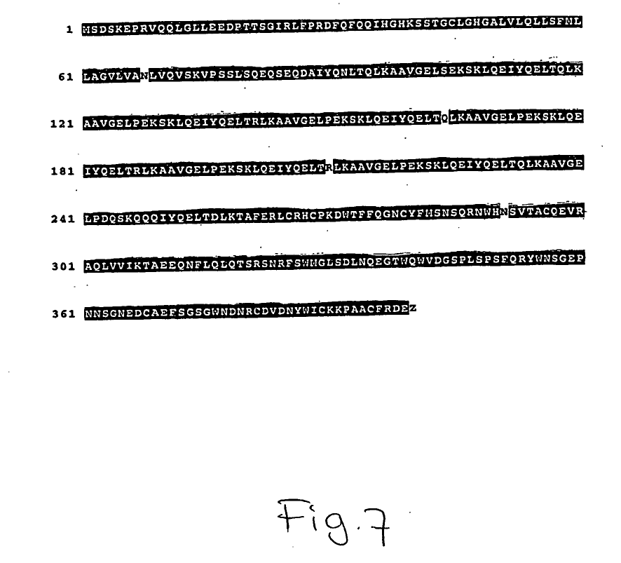

The C-type lectin on the surface of a cell of a sinusoid endothelial layer, in particular a LSEC, is preferably a protein with the amino acid sequence of FIG. 7, or a natural variant or equivalent thereof.

Alternatively, the compound that can bind to a C-type lectin on the surface of a cell of a sinusoid endothelial layer, in particular a LSEC, is a monoclonal antibody, preferably a monoclonal antibody directed against a C-type lectin with the amino acid sequence of FIG. 7 or a natural variant or equivalent thereof; and/or a part, fragment or epitope thereof.

According to a further aspect thereof the invention relates to an antibody, preferably monoclonal antibody, directed against a C-type lectin with the amino acid sequence of FIG. 7 or a natural variant or equivalent thereof; and/or a part, fragment or epitope thereof. This antibody is preferably AZN-D3, which is obtainable by a method as described in the examples.

The invention further relates to a pharmaceutical composition, containing at least one such antibody and at least one carrier, excipient, adjuvant and/or formulant.

Another aspect of the present invention relates to a combination of: 1) a compound that binds to a C-type lectin on the surface of a cell of a sinusoid endothelial layer, in particular a LSEC; and attached thereto: 2) an antigen or a fragment or part thereof. Preferably, the antigen is covalently bonded to or fused with the compound that can bind to the C-type lectin.

In a combination according to the invention the antigen is for example chosen from cancer antigens which can be used to generate an immune response against tumor cells that contain or express said antigen, or antigens as used in vaccines against infectious diseases.

The compound that can bind to a C-type lectin on the surface of a cell of a sinusoid endothelial layer, in particular a LSEC, is preferably chosen from the group consisting of mannose carbohydrates, such as mannan and D-mannose; fucose carbohydrates, such as L-fucose; plant lectins such as concanavalin A; antibiotics, such as pradimicin A; sugars such as N-acetyl-D-glucosamine and galactose; proteins such as gp120 and analogs or fragments thereof; and antibodies directed against a C-type lectin as expressed on the surface of a cell of a sinusoid endothelial layer, in particular a LSEC, or a part, fragment or epitope thereof.

The antibodies of the invention can furthermore be used in the detection of cells of a sinusoid endothelial layer, in particular LSECs, in a biological sample and in the isolation, preparation and/or purification of cells of a sinusoid endothelial layer, in particular LSECs, from a biological sample or a culture medium.

Alternatively, such antibody can find an application in an assay for determining the presence and/or the expression of C-type lectins, in particular a C-type lectin with the amino acid sequence of FIG. 7 or a natural variant or equivalent thereof; and/or a part, fragment or epitope thereof, in a biological sample.

Furthermore, the invention relates to a method for producing, isolating and/or purifying cells of a sinusoid endothelial layer, in particular LSECs, from a biological sample or a culture medium, comprising the steps of:

a) contacting a biological sample or a culture medium that contains said cells with an antibody according to the invention;

b) separating the cells that bind to said antibody from cells that do not bind to said antibody, and optionally from any further constituents of the sample or medium;

and optionally further comprises the step of:

c) separating the cells that bind to the antibody from said antibody.

Preferably, the antibody is attached to a column or matrix, to (para)magnetic beads or to a similar solid support. Biological samples to be tested may be biological fluids such as blood, plasma or lymph fluid.

Finally, the invention provides cells of a sinusoid endothelial layer, in particular LSECs, obtained via the method described above.

The present invention is further illustrated in the example that follows and in which reference is made to the following figures:

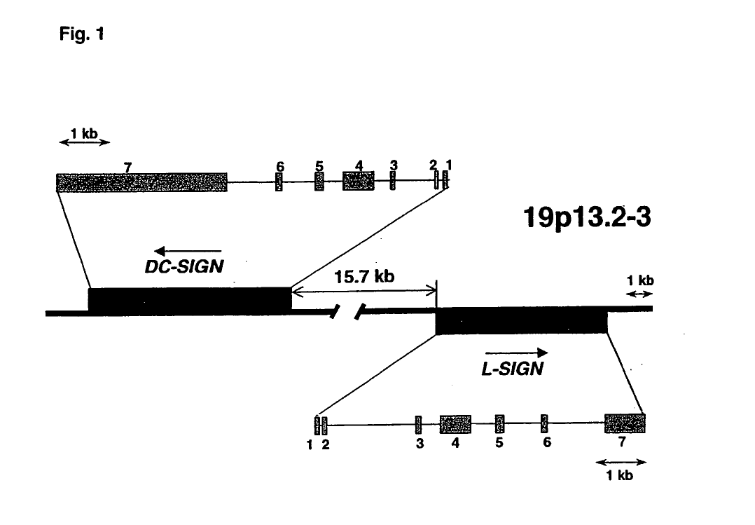

FIG. 1. Schematic representation of the DC-SIGN/L-SIGN genetic map. Physical distances and gene orientation are based on the sequence provided from BAC clone CTD-2102F19 (GenBank AC008812).

FIG. 2. Northern blot analysis of DC-SIGN and L-SIGN. Positions of the 4.3 kb (arrows with solid heads) and 1.9 kb (arrows with open heads) sizes are marked on the left. (A) Hybridization with the L-SIGN-specific probe indicating expression of the gene in liver, lymph node, and weakly in thymus. (B) Hybridization with the probe recognizing both genes. 4.3 kb bands represent DC-SIGN mRNA. The light upper band (^{˜}4.2 kb) evident in liver and lymph node using the L-SIGN-specific probe (FIG. 3A) is distinct from DC-SIGN mRNA (4.3 kb) due to the specificity of the probe, intensity patterns, and slight differences in size. (C) Reprobing of the blots with the β-actin cDNA control probe.

FIG. 3. L-SIGN is expressed on LSECs and not on monocyte-derived DCs. (A) The antibody AZN-D1 is DC-SIGN-specific whereas AZN-D3 cross-reacts with L-SIGN. Stable DC-SIGN and L-SIGN K562 transfectants were stained with either AZN-D1 or AZN-D3. (B) Immunohistochemical analysis of DC-SIGN and L-SIGN expression in the human liver. Serial sections were stained with either AZN-D1 (DC-SIGN-specific) or with AZN-D3 (detects both DC-SIGN and L-SIGN). AZN-D1 stains infrequent cells that may be DCs (arrows), whereas AZN-D3 stains cells lining sinusoids. (C) Expression of L-SIGN in liver is restricted to LSECs. One day after isolation, primary human liver cells were incubated with fluorochrome labeled ovalbumin. L-SIGN expression was determined by indirect immunofluorescence using an L-SIGN-specific polyclonal antibody. Cells that have taken up ovalbumin (LSECs) and those that did not take up ovalbumin (hepatocytes and other resident hepatic cells) are represented by solid and broken lines, respectively, by gating on the respective cell populations. 2×10⁵ cells were analyzed. (D) L-SIGN is not expressed by monocyte-derived DCs. Immature DCs, cultured from monocytes in the presence of GM-CSF and IL-4, do not stain with anti-L-SIGN polyclonal antibody, as determined by FACScan analysis. Solid line indicates staining with anti-L-SIGN polyclonal serum, whereas stippled line (hidden under solid lane) represents staining with rabbit pre-immune serum.

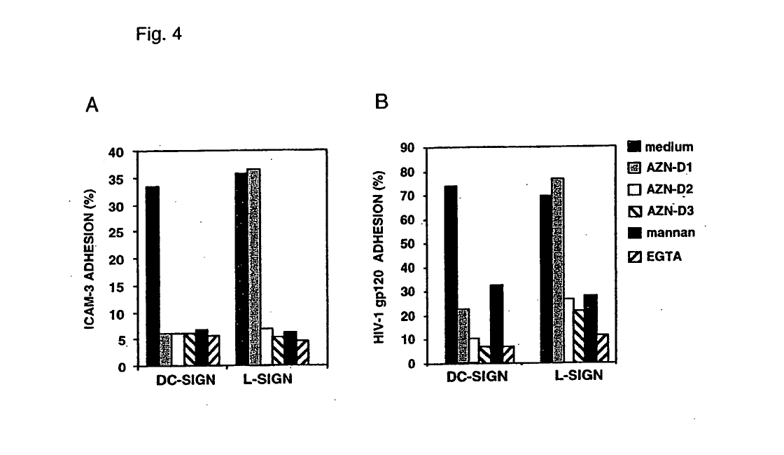

FIG. 4. L-SIGN binds ICAM-3 (A) and HIV-1 gp120 (B). Adhesion of ICAM-3 and gp120 to the K562-L-SIGN and K562-DC-SIGN cells was measured with the fluorescent bead adhesion assay (Geijtenbeek, T. B. et al., 1999, Blood 94:754-764). The y-axis represents the percent cells binding ligand-coated fluorescent beads. The L-SIGN-cross-reacting mAb AZN-D2 (20 μg/ml) and AZN-D3 (20 μg/ml) inhibit the adhesion of ICAM-3 and gp120 to L-SIGN, in contrast to the DC-SIGN-specific mAb AZN-D1 (20 μg/ml). Adhesion of both ICAM-3 and gp120 to the K562 transfectants is also inhibited by either-mannan (20 μg/ml) or EGTA (5 mM). Adhesion of both ligands to mock transfectants was less than 5%. One representative experiment out of three is shown (SD<5%).

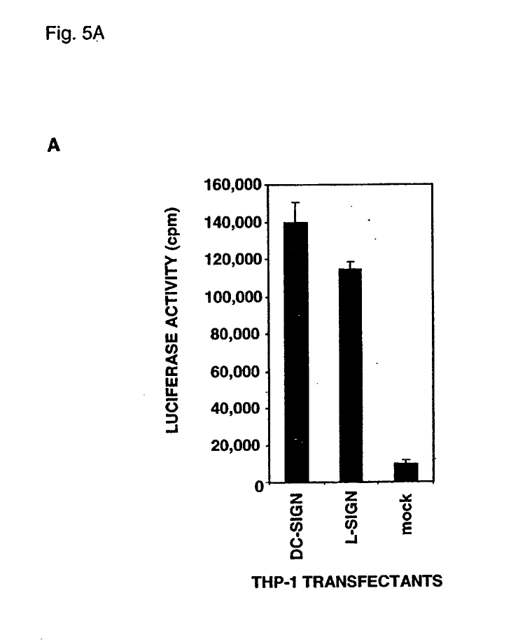

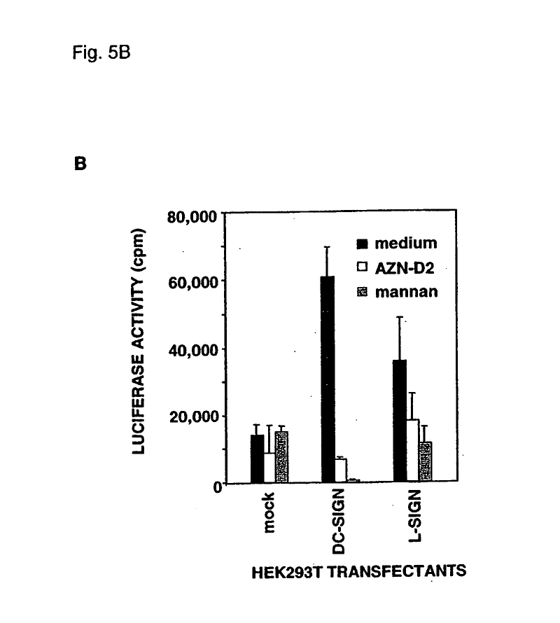

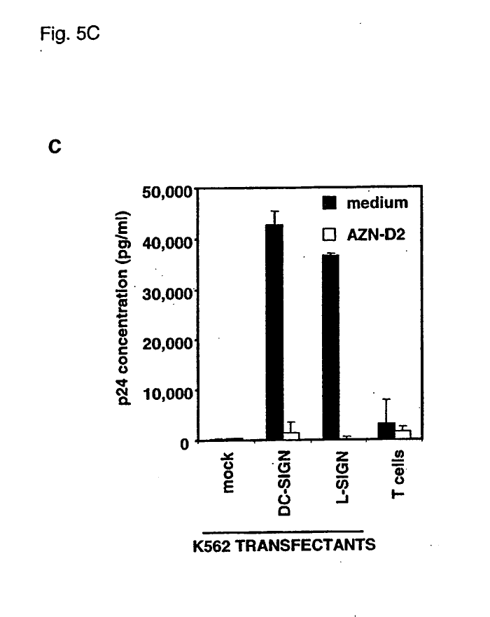

FIG. 5. L-SIGN captures and enhances infection of T cells with HIV-1 in trans. (A) L-SIGN captures HIV-1 and transmits it to target cells. Stable DC-SIGN or L-SIGN expressing THP-1 transfectants were pre-incubated with HIV-luc/JRFL pseudovirions to allow capture of the virus. Cells were washed and THP-1 transfectants were co-cultured with Hut/CCR5 target cells. Cell lysates were obtained after 3 days and analyzed for luciferase activity. For each of the co-culture conditions employed, mock infected controls were uniformly less than 100 counts per second in activity. Each data set represents the mean of four separate wells of infected cells. One representative experiment out of two is shown. (B) L-SIGN enhances infection of T cells by pseudotyped HIV-1. HEK293T cells were transiently transfected with cDNA encoding DC-SIGN, L-SIGN or empty vector. Control cells were preincubated with AZN-D2 (20 μg/ml) or mannan (20 μg/ml). Low amounts of pseudotyped HIV-1_{ADA} were added together with activated T cells as described previously (Geijtenbeek, T. B. et al., 2000, Cell 100: 587-597). Infectivity was determined after two days by measuring luciferase activity. One representative experiment of two performed is shown. Each experiment was done in triplicate wells. (C) L-SIGN enhances infection of T cells by replication competent HIV-1. Stable K562 transfectants of both L-SIGN and DC-SIGN were incubated with low virus concentrations of replication-competent M-tropic strain HIV-1_{JR-CSF} (TCID₅₀ 100/ml). To determine the specificity, cells were preincubated with AZN-D2 (20 μg/ml). After two hours, activated T cells were added as described previously (Geijtenbeek, T. B. et al., 2000, Cell 100:575-585). Culture supernatants were collected at day 14 after K562-T cell co-culture and HIV-1 production was measured using ELISA to determine p24 antigen levels. In control experiments, the same amount of virus was added directly to T cells. One representative experiment out of three is shown. Each data set represents the mean of three separate wells of infected cells.

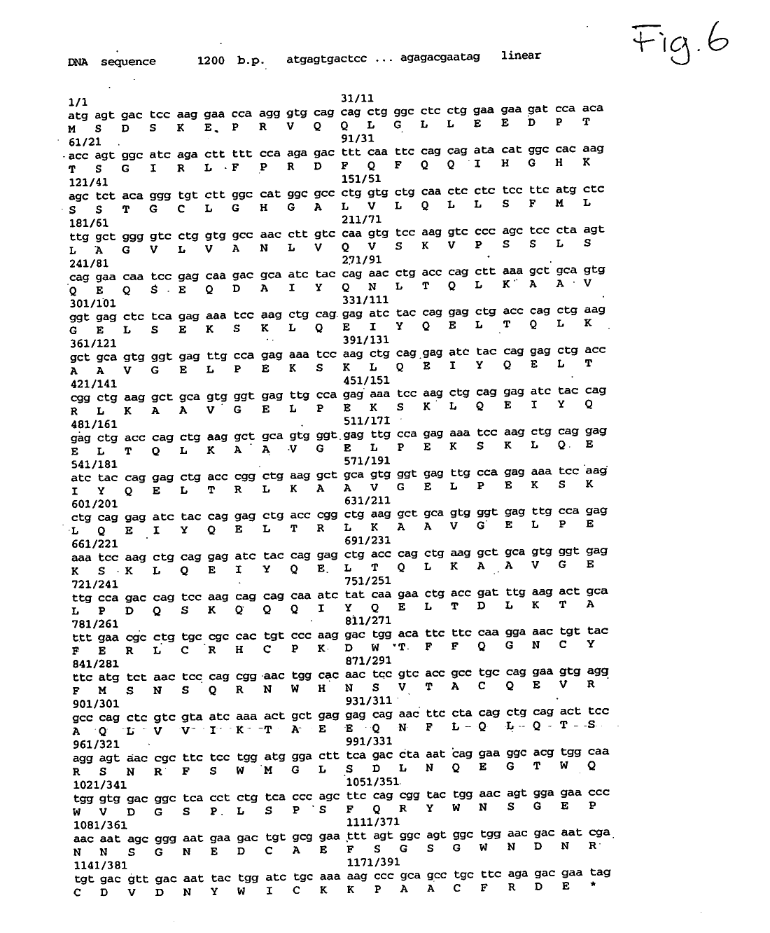

FIG. 6. Coding DNA sequence of L-SIGN

FIG. 7. Amino acid sequence of L-SIGN.

### EXAMPLE

### Materials and Methods

### 1. Characterization of DC-SIGN and LD2 cDNA

The full DC-SIGN and L-SIGN cDNA sequences were submitted to GenBank under accession numbers AF290886 and AF290887, respectively. The L-SIGN cDNA sequence represents a variant containing 6 repeats in exon 4. The 5′ and 3′ ends of the transcripts (except 3′ end of the DC-SIGN mRNA) were determined by 5′RACE (Clontech, Palo Alto, Calif.). The length of the 3′ end of the DC-SIGN mRNA was estimated based on Northern analysis data (transcript size), and RT-PCR data using forward primers specific for the 1.3 kb DC-SIGN cDNA sequence (Curtis, B. M. et al., 1992, Proc Natl Acad Sci USA 89:8356-8360) and reverse primers specific for several GenBank ESTs (e.g. AI472111, AA454170) mapping downstream of alleged 3′ end of DC-SIGN. A cDNA fragment containing the full coding sequence of L-SIGN (nt 39 to 1184, GenBank AF290887) was amplified from human placental mRNA (Clontech) and cloned into the expression vectors pcDNA3.1/V5-His/TOPO (pcDNA3-L-SIGN) and pCDM8 (pCDM8-L-SIGN).

### 2. Radiation Hybrid (RH) Mapping

PCR-based RH mapping with DC-SIGN- and L-SIGN-specific primers was performed using the Genebridge 4 RH panel (Research Genetics, Huntsville, Ala.). The PCR results were submitted to the Gene Map server at the Sanger Center (http://www.sanger.ac.uk/Software/Rhserver). The chromosomal position of markers linked to the genes was determined searching the Genatlas database (http://web.citi2.fr/GENATLAS) and the genetic map of human chromosome 19 provided by the Marshfield Clinic (Marshfield, Wis., http://research.marshfieldclinic.org/genetics/).

### 3. Genotype Analysis of L-SIGN and DC-SIGN Exon 4

The repeat region in exon 4 was amplified with the following pairs of primers:

1) L28, TGTCCAAGGTCCCCAGCTCCC, and L32, GAACTCACCAAATGCAGTCTTCAAATC, for L-SIGN;

2) DL27, TGTCCAAGGTCCCCAGCTCC, and DI4R, CCCCGTGTTCTCATTTCACAG, for DC-SIGN. The cycle conditions were as follows: 94° C. for 5 sec and 68° C. for 1 min. Alleles were distinguished by agarose gel electrophoresis and ethidium bromide staining.

### 4. Northern Blot Analysis

Total RNA from cultured human immature DCs (see below) was isolated using Trizol (Life Technologies, Rockville, Md.). Ten μg of the isolated RNA were electrophoresed on a 1% agarose gel, transferred to Hybond-XL (Amersham Pharmacia Biotech, Backinghamshire, England) as described (Chomczynski, P. 1992. Anal Biochem 201:134-139), and used for Northern analysis along with two human multiple tissue Northern blots (Clontech). Three probes were subsequently hybridized to the blots:

1) an L-SIGN-specific probe (nt 100 to 183, GenBank AF290887),

2) a probe recognizing both DC-SIGN and L-SIGN (nt 1 to 1233, GenBank AF290886) and

3) an actin control probe (Clontech).

Hybridization procedures were performed according to manufacturer specifications (Clontech).

### 5. Antibodies

Anti-DC-SIGN mAb AZN-D1 and AZN-D2 were described previously (Geijtenbeek, T. B. et al., 2000b, Cell 100:575-585). mAb AZN-D3 was obtained by screening hybridoma supernatants of BALB/c mice immunized with THP-1-DC-SIGN cells (Geijtenbeek, T. B. et al., 2000a, Cell 100:587-597) for the ability to stain both DC-SIGN and L-SIGN. Anti-DC-SIGN mAb AZN-D2 also cross-reacts with L-SIGN, as was initially determined by staining of K562-L-SIGN cells (data not shown). Anti-L-SIGN rabbit antiserum was generated by immunization with two L-SIGN-specific peptides, PTTSGIRLFPRD and WNDNRCDVDNYW (Veritas, Inc. Laboratories, Rockville, Md.).

### 6. Cells

DCs were cultured from monocytes in the presence of 500 U/ml IL4 and 800 U/ml GM-CSF (Schering-Plough, Brussels, Belgium) (Sallusto, F., and A. Lanzavecchia. 1994. J Exp Med 179:1109-1118; Romani, N. et al., 1994, J Exp Med 180:83-93). At day 7 the cells expressed high levels of MHC class I and II, αMβ2 (CD11b), αXβ2 (CD11c), DC-SIGN and ICAM-1, moderate levels of LFA-1 and CD86, and low levels of CD14, as measured by flow cytometry. Stable K562 transfectants expressing L-SIGN (K562-L-SIGN) were generated by co-transfection of K562 with the pCDM8-L-SIGN plasmid and the pGK-neo vector by electroporation (Lub, M. et al., 1997, Mol Biol Cell 8:719-728). Stable K562-DC-SIGN transfectants were generated in a similar manner using pRc/CMV-DC-SIGN (2). THP-1-DC-SIGN cells were described previously (Geijtenbeek, T. B. et al., 2000b, Cell 100:575-585).

Stable THP-1-L-SIGN transfectants were generated by electroporation of THP-1 cells with pcDNA3-L-SIGN, selection for G418-resistance, and positive sorting for L-SIGN expression using mAb AZN-D3. All cell lines were maintained in RPMI medium supplemented with 10% fetal bovine serum in addition to specific cytokine or antibiotic requirements as indicated. K562 and THP-1 are monocytic cell lines.

HEK293T are human embryic kidney cell containing a single temperature-sensitive allele of SV-40 large T antigen. GHOST cells are HIV-indicator cells derived from human osteosarcoma cells (Cecilia, D. et al., 1998, J Virol 72:6988-6996). Hut/CCR5 cells are the transformed human T cell line Hut78 stably transduced with CCR5.

### 7. Fluorescent Beads Adhesion Assay

Carboxylate-modified TransFluorSpheres (488/645 nm, 1.0 μm; Molecular Probes, Eugene, Oreg.) were coated with ICAM-3 as was previously described for ICAM-1 (Geijtenbeek, T. B. et al., 1999, Blood 94:754-764). Fluorescent beads were coated with M-tropic HIV-1_{MN} envelope glycoprotein gp120 as follows: Streptavidin-coated fluorescent beads were incubated with biotinylated F(ab′)2 fragment rabbit anti-sheep IgG (6 μg/ml; Jackson Immunoresearch) followed by an overnight incubation with sheep-anti-gp120 antibody D7324 (Aalto Bio Reagents Ltd, Dublin, Ireland) at 4° C. The beads were washed and incubated with 250 ng/ml purified HIV-1 gp120 (provided by Immunodiagnostics, Inc through the NIH AIDS Research and Reference Reagent Program) overnight at 4° C.

The fluorescent beads adhesion assay was performed as described by Geijtenbeek et al.(1999, supra). Briefly, cells were resuspended in adhesion buffer (20 mM Tris-HCl pH 8.0, 150 mM NaCl, 1 mM CaCl₂, 2 mM MgCl₂, 0.5 BSA) at a final concentration of 5×10⁶ cells/ml. 50,000 cells were pre-incubated with mAb (20 μg/ml) for 10 min at room temperature. Ligand-coated fluorescent beads (20 beads/cell) were added and the suspension was incubated for 30 min at 37° C. Adhesion was determined by measuring the percentage of cells that bound fluorescent beads using flow cytometry on a FACScan (Becton Dickinson, Oxnard, Calif.).

### 8. Detection of L-SIGN on Primary Human Liver Sinusoidal Endothelial Cells (LSECs)

Liver tissue was obtained from a patient undergoing liver surgery after having received written consent. Isolation of primary human liver cells was performed as previously described (Hegenbarth, S. et al., 2000, Hum Gene Ther 11:481-486). Cells were cultured on collagen type I coated tissue culture plates in supplemented Williams E Medium (Hild, M. et al., 1998, J Virol 72:2600-2606). The day after isolation, liver cells were incubated with Texas-Red labelled ovalbumin (10 μg/ml) (Molecular Probes, Leiden, Netherlands) for two hours and detached from the matrix by gentle trypsin treatment. Cells were stained with rabbit anti-L-SIGN antiserum followed by goat-anti-rabbit-Ig FITC (Dianova, Hamburg, Germany) and analyzed with a FACScan (Becton Dickinson, Heidelberg, Germany) using CellQuest software. Ovalbumin uptake was characteristic of LSECs only and not Kupffer cells, as verified by co-staining of ovalbumin-positive cells with an endothelial cell-specific marker, acetylated LDL, using confocal microscopy.

### 9. HIV-1 Infection Assays

The infection assays were performed as described previously (Geijtenbeek, 2000a, supra; Geijtenbeek 2000b, supra). Pseudotyped HIV-1 stocks were generated by calcium-phosphate transfections of HEK293T cells with the proviral vector plasmid NL-Luc-E⁻R⁻containing a firefly luciferase reporter gene (Connor, R. I. et al., 1995, Virology 206:935-944) and expression plasmids for either ADA or JRFL gp160 envelopes. Viral stocks were evaluated by limiting dilution on GHOST CXCR4/CCR5 and 293T-CD4-CCR5 cells.

In HIV-1 cell capture assays, DC-SIGN or L-SIGN expressing THP-1 transfectants (250,000 cells) were pre-incubated with pseudotyped HIV-1 (multiplicity of infection ^{˜}0.1 with regard to target cell concentration) in a total volume of 0.5 ml for 3 hr to allow cellular adsorption of the virus. After the 3 hr incubation, cells were washed with 2 volumes PBS and the THP-1 transfectants were co-cultured with Hut/CCR5 targets (100,000 cells) in the presence of 10 μg/ml polybrene in 1 ml cell culture medium. Cell lysates were obtained after 3 days and analyzed for luciferase activity.

In contrast, HIV-1 enhancement assays utilized suboptimal concentrations of virus (typically <0.05 m.o.i.) without a wash step. Briefly, DC-SIGN or L-SIGN transfectants (50,000 cells) were incubated with identical virus concentrations (either pseudotyped HIV-1 or replication-competent M-tropic strain HIV-1_{JR-CSF}), and after 2 hr activated T cells (100,000 cells) were added. Cell lysates were obtained after several days and analyzed for either luciferase activity or p24 antigen levels. T cells were activated by culturing them in the presence of IL-2 (10 U/ml) and PHA (10 μg/ml) for 2 days.

### 10. Immunohistochemical Analysis

Staining of the tissue cryosections was performed as described previously (Geijtenbeek, 2000b, supra). Cryosections (8μ) of the tissues were fixed in 100% acetone (10 min), washed with PBS and incubated with the first antibody (10 μg/ml) for 60 min at 37° C. After washing, the final staining was performed with the ABC-PO/ABC-AP Vectastain kit (vector Laboratories, Burlingame, Calif.) according to the manufacturer's protocol. Nuclear staining was performed with hematoxylin.

### Results

### 1. Genomic Map of DC-SIGN and L-SIGN

A fine map of the DC-SIGN/L-SIGN gene locus was determined using information from the human BAC clone CTD-2102F19 sequence, which is now available in GenBank (AC008812) (FIG. 1). DC-SIGN and L-SIGN are positioned in a head-to-head orientation 15.7 kb apart. RH mapping indicated that DC-SIGN and L-SIGN are located on chromosome 19p13.2-3 near the marker D19S912 (lod score values >11.1) with DC-SIGN positioned more telomeric. In agreement with the RH daeta, the D19S912 marker is found at a distance of about 37 kb centromeric to L-SIGN on the BAC sequence.

### 2. Polymorphism in Exon 4 of L-SIGN

Exon 4 of both DC-SIGN and L-SIGN contain repeats of 69 bp that encode repeating units of 23 amino acids. These repeats form a neck between the carbohydrate recognition domain and the transmembrane domain of the SIGN molecules. The L-SIGN cDNA clone isolated from placental mRNA contained the entire coding region of the gene, but only 6 full repeats were present in the sequence corresponding to exon 4, in contrast to 7 full repeats identified in the cDNA reported by Soilleux et al. (2000, supra). This indicated that the repeat region of L-SIGN is polymorphic. Analysis of exon 4 in 350 Caucasian individuals showed the presence of seven alleles based on number of repeats (ranging from 3 to 9), the most common of which was the allele containing 7 repeats (Table 1). Analysis of DC-SIGN exon 4 in 150 Caucasians did not reveal any variability.

### 3. Northern Analysis of DC-SIGN and L-SIGN

L-SIGN mRNA exhibits about 90% similarity to DC-SIGN mRNA over the entire coding region, but there is only 53% similarity between exons 2 of the genes. Therefore, exon 2 sequence was used to generate a probe (84 nt) that was L-SIGN specific in Northern analysis. The probe hybridized to mRNA of about 1.9, 2.6 and 4.2 kb in size in liver and lymph node, and a weak 1.9 kb band was detected in thymus (FIG. 2A). The 1.9 kb band, which is prominent in lymph node and fetal liver, corresponds to the predicted size of L-SIGN. The upper bands (one of which, 2.6 kb, is substantial in adult liver) are likely to be alternative transcripts, but RACE and RT-PCR techniques have not indicated the presence of untranslated regions varying in length nor alternative splice variants.

Northern blots were reprobed with a 1.2 kb fragment containing the entire coding sequence of DC-SIGN, which recognizes both DC- and L-SIGN mRNA due to their high sequence similarity (FIG. 2B). Once again, the bands representing L-SIGN transcripts were observed in liver, lymph node and fetal liver. Additionally, a 4.3 kb transcript representing DC-SIGN was detected in monocyte-derived DCs and lymph node, and to a lesser extent in placenta, spleen, thymus, and possibly, liver.

L-SIGN mRNA was also detected in placenta and DCs using a more sensitive RT-PCR technique, but the level of expression in these tissues is too low to be detected by Northern hybridization. The probe which recognizes both DC-SIGN and L-SIGN transcripts with nearly equal sensitivity clearly indicated differential tissue distribution of the two gene products: L-SIGN is primarily transcribed in liver and lymph node, whereas DC-SIGN is specifically expressed in DCs and in tissues that accommodate DCs (FIG. 2). L-SIGN mRNA is not detected by Northern analysis in DCs, peripheral blood lymphocytes, nor spleen (FIG. 2).

### 4. L-SIGN is Expressed by Human LSECs and Not by DCs

To identify the cells expressing L-SIGN molecules in vivo, immunohistochemical analysis was performed using a pair of anti-DC-SIGN mAbs, one of which, AZN-D3, cross-reacted with L-SIGN, whereas another, AZN-D1, was DC-SIGN-specific (FIG. 3A). As expected from the Northern analysis, poor staining of liver tissue was observed using the DC-SIGN-specific mAb AZN-D1 (FIG. 3B) and the rare cells detected with this antibody are probably DCs residing in liver. In contrast, the mAb AZN-D3 brightly stained cells lining the sinusoids of the liver (FIG. 3B).

Monoclonal antibodies against the endothelial cell-specific marker CD31 gave a similar staining pattern on serial liver sections (data not shown), showing that L-SIGN is expressed by LSECs. To support this idea, primary human LSECs were distinguished from the other hepatic cells by uptake of ovalbumin, which is a unique characteristic of LSECs, and were tested for expression of L-SIGN directly. Staining of LSECs with polyclonal anti-L-SIGN antibodies indicated that L-SIGN is expressed exclusively by these cells in liver (FIG. 3C).

Both AZN-D1 and AZN-D3 stained lymph node equally well (data not shown). However, using L-SIGN-specific polyclonal antibodies, we found that L-SIGN is not expressed by monocyte-derived DCs (FIG. 3D), which supports conclusions from the Northern analysis. DC-SIGN and L-SIGN are expressed by different types of cells in the lymph node.

### 5. L-SIGN Binds ICAM-3 and HIV-1 qp120

Both ICAM-3 and HIV-1_{MN} gp120 have been shown to bind-with high affinity to DC-SIGN in a Ca²⁺ dependent manner. Using a flow cytometry-based adhesion assay (Geijtenbeek, 1999, supra), K562 cells transfected with L-SIGN were shown to bind. ICAM-3 with high affinity (FIG. 4A). The L-SIGN-mediated binding was inhibited by the DC-SIGN/L-SIGN-specific mAb AZN-D2 and AZN-D3, mannan, or EGTA, but not by the DC-SIGN-specific mAb AZN-D1, demonstrating that L-SIGN functions as a mannose binding C-type lectin with a high affinity for ICAM-3. L-SIGN was also able to bind to HIV-_{MN} gp120 (FIG. 4B). Mock transfected cells did not bind either ICAM-3 or HIV-1_{MN} gp-120 (data not shown).

### 6. L-SIGN Enhances HIV-1 Infection

High affinity binding of L-SIGN to HIV-1 gp120 raised the possibility that, L-SIGN might bind infectious HIV-1 and enhance infection of target cells in trans. To test the role of L-SIGN as a trans-receptor in HIV-1 infection, THP-1 cells expressing either-DC-SIGN or L-SIGN were pulsed with single-round infectious HIV-luciferase pseudotyped with M-tropic HIV-1_{JRFL} envelope glycoprotein, washed to remove unbound virus, and incubated with target cells permissive for HIV-1 infection. Infection was evaluated after three days. Both the L-SIGN- and DC-SIGN-transfected THP-1 cells captured infectious HIV-1 and transmitted the virus to target cells, while mock transfected THP-1 cells did not (FIG. 5A).

Next it was investigated whether L-SIGN would be able to capture a limiting concentration of HIV-1 and efficiently present the virus to the permissive cells promoting infection. HEK293T cells expressing DC-SIGN or L-SIGN, or mock transfected cells were incubated with low titers of HIV-luciferase pseudoptyped with HIV-1_{ADA} envelope glycoprotein. The unwashed cells were then co-cultured with activated T cells. Minimal infection of target cells was observed from mock transfected HEK293T cells pulsed with HIV-1 (FIG. 5B). However, HEK293T cells transfected with L-SIGN enhanced HIV-1 infection of T cells in trans (FIG. 5B). The DC-SIGN-mediated enhancement was inhibited with the crossreactive AZN-D2 antibody, while partial inhibition was observed for L-SIGN. Mannan efficiently inhibited enhancement by both SIGN molecules.

Similar experiments to evaluate the ability of L-SIGN to enhance HIV-1 infection of T-cells were performed using replication competent virus. K562 cells transfected with L-SIGN, DC-SIGN, and empty vector were incubated with the M-tropic HIV-1_{JR-CSF} strain at low virus concentrations for 2 hours and subsequently co-cultured with activated T cells (FIG. 5C). No viral replication was observed using mock transfected K562 cells, while L-SIGN transfectants transmitted HIV-1 to target cells, resulting in viral replication. Almost complete inhibition of HIV-1 replication with the DC-SIGN/L-SIGN-specific antibody AZN-D2 indicated the specificity of these receptors to enhance HIV-1 infection. Thus, non-DC lineage cells expressing L-SIGN within liver and possibly in lymph node also have the ability to capture and transmit HIV-1 to lymphocytes.

TABLE 1
Polymorphism of the repeat region in L-SIGN exon 4.

| No. of repeats | Allele frequency (%) | |
|---|---|---|
| 3 | 1 | (0.3) |
| 4 | 25 | (3.6) |
| 5 | 202 | (28.9) |
| 6 | 86 | (12.2) |
| 7 | 377 | (53.9) |
| 8 | 2 | (0.3) |
| 9 | 7 | (1.0) |

1. Use of a compound that binds to a C-type lectin on the surface of a sinusoid endothelial layer, in the preparation of a composition for modulating, in particular reducing, the immune response in a animal, in particular a human or another mammal.

2. Use according to claim 1, wherein the sinusoid endothelial layer is constituted by liver sinusoid endothelial cells (LSEC).

3. Use according to claim 1, wherein the sinusoid endothelial layer is constituted by the lymph node sinusoidal zone.

4. Use according to claim 1 and/or 2, in the preparation of a composition for modulating, in particular reducing, one or more interactions between a cell of a sinusoid endothelial layer, in particular a LSEC, and a cell expressing ICAM-2 or ICAM-3, in particular a T cell.

5. Use according to claim 1 and/or 2 and/or 4, in the preparation of a composition for modulating, in particular reducing, the adhesion between a cell of a sinusoid endothelial layer, in particular a LSEC, and a cell expressing ICAM-2 or ICAM-3, in particular a T cell, in particular between a C-type lectin on the surface of a LSEC and an ICAM receptor on the surface of a cell expressing ICAM-2 or ICAM-3, in particular a T cell, in particular an ICAM-2 or ICAM-3 receptor on the surface of a T cell.

6. Use according to any of claims 1-5, in the preparation of a composition for preventing or inhibiting immune responses to specific antigens, for inducing tolerance, for immunotherapy, for immunosuppression, for the treatment of autoimmune diseases, and/or for the treatment of allergy.

7. Use of a compound that binds or can bind to a C-type lectin on the surface of a cell of the sinusoid endothelial layer, in particular a LSEC, in the preparation of a composition for-inhibiting the HIV infection of cells of a sinusoid endothelial layer, in particular LSECs, in particular for inhibiting the adhesion of HIV surface protein (i.e gp 120) to the surface of a cell of a sinusoid endothelial layer, in particular a LSEC and thereby the entry of HIV into said cell.

8. Use of a compound that binds or can bind to a C-type lectin on the surface of a cell of a sinusoid endothelial layer, in particular a LSEC, in the preparation of a composition for inhibiting the transfer of HIV from cells of a sinusoid endothelial layer, in particular a LSEC, to non-infected T cells.

9. Use of a combination of: 1) a compound that binds to a C-type lectin on the surface of a cell of a sinusoid endothelial layer, in a particular a LSEC; and attached thereto: 2) an antigen or a fragment or part thereof; in the preparation of a composition for modulating, in particular generating, increasing and/or promoting, an immune response in an animal, in particular a human or other mammal, against said antigen.

10. Use according to claim 9, in which the antigen is covalently bonded to or fused with the compound that can bind to the C-type lectin.

11. Use according to claim 9 or 10, in which the antigen is chosen from cancer antigens which can be used to generate an immune response against tumor cells that contain or express said antigen, or antigens as used in vaccines against infectious diseases.

12. Use according to any of claims 1-11, in which the compound that can bind to a C-type lectin on the surface of a cell of a sinusoid endothelial layer, in particular a LSEC, is chosen from the group consisting of mannose carbohydrates, such as mannan and D-mannose; fucose carbohydrates, such as L-fucose; plant lectins such as concanavalin A; antibiotics, such as pradimicin A; sugars such as N-acetyl-D-glucosamine and galactose; proteins such as gp120 and analogs or fragments thereof; and antibodies directed against a C-type-lectin as expressed on the surface of a cell of a sinusoid endothelial layer, in particular a LSEC, or a part, fragment or epitope thereof.

13. Use according to any of claims 1-11 in which the C-type lectin on the surface of a cell of a sinusoid endothelial layer, in particular a LSEC, is a protein with the amino acid sequence of FIG. 7, or a natural variant or equivalent thereof.

14. Use according to claim 12 or 13, in which the compound that can bind to a C-type lectin on the surface of a cell of a sinusoid endothelial layer, in particular a LSEC, is a monoclonal antibody, preferably a monoclonal antibody directed against a C-type lectin with the amino acid sequence of FIG. 7 or a natural variant or equivalent thereof; and/or a part, fragment or epitope thereof.

15. Antibody, preferably monoclonal antibody, directed against a C-type lectin with the amino acid sequence of FIG. 7 or a natural variant or equivalent thereof; and/or a part, fragment or epitope thereof.

16. Antibody according to claim 15, which is AZN-D3.

17. Pharmaceutical composition, containing at least one antibody according to claim 15 or 16, and at least one carrier, excipient, adjuvant and/or formulant.

18. Combination of: 1) a compound that binds to a C-type lectin on the surface of a cell of a sinusoid endothelial layer, in particular a LSEC; and attached thereto: 2) an antigen or a fragment or part thereof.

19. Combination according to claim 18, wherein the antigen is covalently bonded to or fused with the compound that can bind to the C-type lectin.

20. Combination according to claim 18 or 19, in which the antigen is chosen from cancer antigens which can be used to generate an immune response against tumor cells that contain or express said antigen, or antigens as used in vaccines against infectious diseases.

21. Combination according to any of claims 18-20, in which the compound that can bind to a C-type lectin on the surface of a cell of a sinusoid endothelial layer, in particular a LSEC, is chosen from the group consisting of mannose carbohydrates, such as mannan and D-mannose; fucose carbohydrates, such as L-fucose; plant lectins such as concanavalin A; antibiotics, such as pradimicin A; sugars such as N-acetyl-D-glucosamine and galactose; proteins such as gp120 and analogs or fragments thereof; and antibodies directed against a C-type lectin as expressed on the surface of a cell of a sinusoid endothelial layer, in particular a LSEC, or a part, fragment or epitope thereof.

22. Use of an antibody according to claim 15 or 16 in the detection of cells of a sinusoid endothelial layer, in particular LSECs, in a biological sample.

23. Use of an antibody according to claim 15 or 16 in the isolation, preparation and/or purification of cells of a sinusoid endothelial layer, in particular LSECs, from a biological sample or a culture medium.

24. Use of an antibody according to claim 15 or 16 in an assay for determining the presence and/or the expression of C-type lectins, in particular a C-type lectin with the amino acid sequence of FIG. 6 or a natural variant or equivalent thereof; and/or a part, fragment or epitope thereof, in a biological sample.

25. Method for producing, isolating and/or purifying cells of a sinusoid endothelial layer, in particular LSECs, from a biological sample or a culture medium, comprising the steps of:
a) contacting a biological sample or a culture medium that contains said cells with an antibody according to claim 15 or 16;
b) separating the cells that bind to said antibody from cells that do not bind to said antibody, and optionally from any further constituents of the sample or medium;
and optionally further comprises the step of:
c) separating the cells that bind to the antibody from said antibody.

26. Method according to claim 25, in which the antibody is attached to a column or matrix, to (para)magnetic beads or to a similar solid support.

27. Method according to claim 25 or 26, in which the biological sample is a biological fluid such as blood, plasma or lymph fluid.

28. Cells of a sinusoid endothelial layer, in particular LSECs obtained via the method of claims 25 or 26.


SEQUENCE LISTING (6 sequences)

SEQ ID NO: 1 - DNA, 1200 bp, Homo sapiens
  CDS (1)..(1197)
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccaaccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacaactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 2 - protein, 399 aa, Homo sapiens
MSDSKEPRVQQLGLLEEDPTTSGIRLFPRDFQFQQIHGHKSSTGCLGHGALVLQLLSFMLLAGVLVANLVQVSKVPSSLSQEQSEQDAIYQNLTQLKAAVGELSEKSKLQEIYQELTQLKAAVGELPEKSKLQEIYQELTRLKAAVGELPEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHNSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 3 - DNA, 21 bp, Artificial
  Primer L28
tgtccaaggtccccagctccc

SEQ ID NO: 4 - DNA, 27 bp, Artificial
  Primer L32
gaactcaccaaatgcagtcttcaaatc

SEQ ID NO: 5 - DNA, 20 bp, Artificial
  Primer DL27
tgtccaaggtccccagctcc

SEQ ID NO: 6 - DNA, 21 bp, Artificial
  Primer D14R
ccccgtgttctcatttcacag